(19) United States
(12) Patent Application Publication
(10) Pub. No.: US 2020/0103372 A1
(43) Pub. Date: Apr. 2, 2020

(54) ELECTROPHORESIS ANALYZING APPARATUS, ELECTROPHORESIS ANALYSIS METHOD, AND PROGRAM

(71) Applicant: NEC CORPORATION, Tokyo (JP)

(72) Inventor: Minoru ASOGAWA, Tokyo (JP)

(73) Assignee: NEC CORPORATION, Tokyo (JP)

(21) Appl. No.: 16/497,635

(22) PCT Filed: Mar. 28, 2018
(86) PCT No.: PCT/JP2018/012657

§ 371 Date: Sep. 25, 2019

(30) Foreign Application Priority Data
Mar. 29, 2017 (JP) 2017-066161

(51) Int. Cl. G01N 27/447; G06N 7/00; G16B 5/00; G01N 33/487

(52) U.S. Cl. CPC G01N 27/44791; G01N 33/48721; G16B 5/00; G06N 7/005

(57) ABSTRACT

An electrophoresis analyzing apparatus includes an acquisition part, an estimation part, and a correction part. The acquisition part acquires actual waveform data on electrophoresis including at least two peak waveforms partially including a superimposed portion. The estimation part estimates, based on an already-appeared peak waveform, a residual portion of an already-appeared peak waveform in the superimposed portion, the already-appeared peak waveform having appeared, in the actual waveform data, before an analysis-target peak waveform to be subjected to waveform analysis. The correction part subtracts the residual portion from the superimposed portion and corrects the analysis-target peak waveform to obtain a true analysis-target waveform.

### CROSS-REFERENCE TO RELATED APPLICATIONS

This application is a National Stage of International Application No. PCT/JP2018/012657 filed Mar. 28, 2018, claims priority based on Japanese Patent Application No. 2017-066161 (filed on Mar. 29, 2017), the contents of which application are incorporated herein in its entirety by reference.

### TECHNICAL FIELD

The present invention relates to an electrophoresis analyzing apparatus, an electrophoresis analysis method, and a program.

### BACKGROUND

An electrophoresis apparatus is used to analyze a specimen such as a small amount of protein, deoxyribonucleic acid (DNA), or the like (refer to Patent Literature 1). Moreover, there exists a technique for determining the quantity of a specimen, based on actual waveform data of an electropherogram acquired through electrophoresis. For example, in Patent Literature 2, the area of a peak waveform appearing in actual waveform data is calculated to thereby determine the quantity of a specimen.

### Patent Literature 1:

Japanese Patent Kokai Publication No. JP2002-310989A

### Patent Literature 2:

Japanese Patent Kokai Publication No. JP2016-33492A

### SUMMARY

Note that the disclosures in the above-mentioned CITATION LIST are incorporated herein by reference. The following analysis has been made by the inventor of the present invention.

The technique disclosed in Patent Literature 2 described above has a problem that it is not possible to determine the quantity of a specimen when actual waveform data includes at least two peak waveforms partially including a superimposed portion. Specifically, actual waveform data expresses a waveform of a superimposed portion as a total value of first and second peak waveforms, and hence it is not possible to calculate the area of each of the first and second peak waveforms.

The present invention has a primary object to provide an electrophoresis analyzing apparatus, an electrophoresis analysis method, and a program for contributing to improving accuracy of electropherogram analysis.

According to a first aspect of the present invention or disclosure, provided is an electrophoresis analyzing apparatus including: an acquisition part configured to acquire actual waveform data of electrophoresis, the actual waveform data including at least two peak waveforms partially including a superimposed portion; an estimation part configured to estimate, from an already-appeared peak waveform, a residual portion of the already-appeared peak waveform in the superimposed portion, the already-appeared peak waveform having appeared, in the actual waveform data, before an analysis-target peak waveform to be subjected to waveform analysis; and a correction part configured to subtract the residual portion from the superimposed portion to correct the analysis-target peak waveform and obtain a true analysis-target waveform.

According to a second aspect of the present invention or disclosure, provided is an electrophoresis analysis method including: acquiring actual waveform data of electrophoresis, the actual waveform data including at least two peak waveforms partially including a superimposed portion; estimating, from an already-appeared peak waveform, a residual portion of the already-appeared peak waveform in the superimposed portion, the already-appeared peak waveform having appeared, in the actual waveform data, before an analysis-target peak waveform to be subjected to waveform analysis; and subtracting the residual portion from the superimposed portion to correct the analysis-target peak waveform to obtain a true analysis-target waveform.

According to a third aspect of the present invention or disclosure, provided is a program causing a computer to execute: processing of acquiring actual waveform data of electrophoresis, the actual waveform data including at least two peak waveforms and partially including a superimposed portion; processing of estimating, from an already-appeared peak waveform, a residual portion of the already-appeared peak waveform in the superimposed portion, the already-appeared peak waveform having appeared, in the actual waveform data, before an analysis-target peak waveform to be subjected to waveform analysis; and processing of subtracting the residual portion from the superimposed portion and correcting the analysis-target peak waveform to obtain a true analysis-target waveform.

Note that this program may be recoded on a computer-readable storage medium. The storage medium may be a non-transient medium, such as a semiconductor memory, a hard disk, a magnetic recording medium, or an optical recording medium. The present invention may be implemented as a computer program product.

According to the aspects of the present invention or disclosure, an electrophoresis analyzing apparatus, an electrophoresis analysis method, and a program for contributing to improving accuracy of electropherogram analysis are provided.

### BRIEF DESCRIPTION OF THE DRAWINGS

FIG. 1 is a diagram for illustrating an outline of one example embodiment.

FIGS. 2A to 2C are graphs for illustrating the outline of the one example embodiment.

FIG. 3 is a diagram illustrating an example of a schematic configuration of an electrophoresis system according to a first example embodiment.

FIG. 4 is a diagram illustrating a correspondence relationship between fluorescence intensity and elapsed electrophoresis time.

FIG. 5 is a diagram illustrating an example of a processing configuration of an electrophoresis analyzing apparatus according to the first example embodiment.

FIGS. 6A and 6B are graphs illustrating an example of a signal strength waveform.

FIGS. 7A and 7B are diagrams for illustrating occurrence of a superimposed portion.

FIGS. 8A and 8B are diagrams for illustrating the occurrence of the superimposed portion.

FIGS. 9A and 9B are diagrams for illustrating the occurrence of the superimposed portion.

FIGS. 10A to 10C are diagrams for illustrating the occurrence of the superimposed portion.

FIGS. 11A and 11B are diagrams for illustrating the occurrence of the superimposed portion.

FIGS. 12A to 12C are graphs for illustrating operations of a residual amount estimation part.

FIG. 13 is a flowchart illustrating an example of operations of the electrophoresis analyzing apparatus according to the first example embodiment.

FIG. 14 is a block diagram illustrating an example of a hardware configuration of the electrophoresis analyzing apparatus according to the first example embodiment.

### PREFERRED MODES

First of all, an outline of one example embodiment is described. Note that the reference signs in the drawings added in this outline are given, as an example, to elements for convenience for the sake of better understanding, and the description of this outline is not intended to provide any particular limitation.

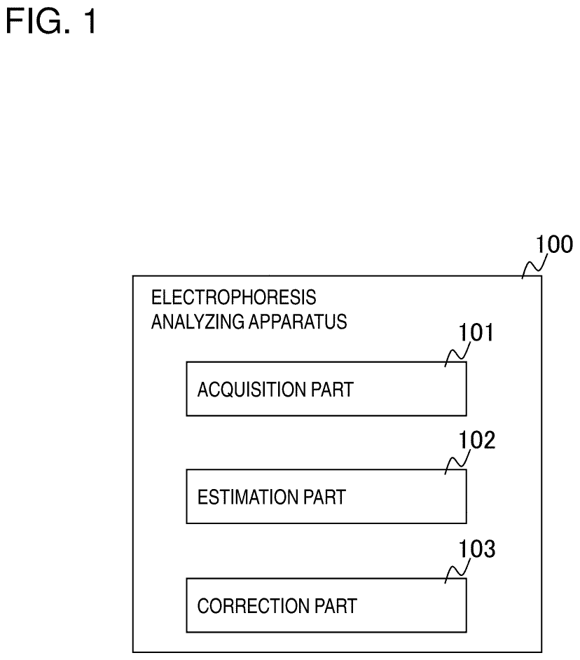

As illustrated in FIG. 1, an electrophoresis analyzing apparatus 100 according to the one example embodiment includes an acquisition part 101, an estimation part 102, and a correction part 103. The acquisition part 101 acquires actual waveform data on electrophoresis including at least two peak waveforms partially including a superimposed portion. The estimation part 102 estimates, based on an already-appeared peak waveform, a residual portion of the already-appeared peak waveform in the superimposed portion, the already-appeared peak waveform having appeared before an analysis-target peak waveform to be subjected to waveform analysis in the actual waveform data. The correction part 103 subtracts the residual portion from the superimposed portion to correct the analysis-target peak waveform to obtain a true analysis-target waveform.

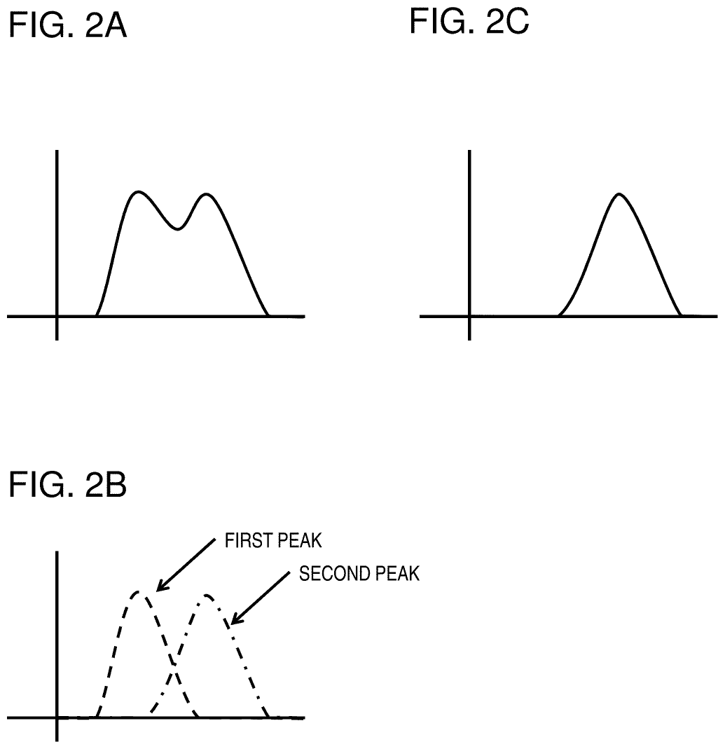

The acquisition part 101 acquires actual waveform data on electrophoresis as one illustrated in FIG. 2A. The actual waveform data illustrated in FIG. 2A presents a waveform in which first and second peak waveforms illustrated in FIG. 2B are partially superimposed on each other. The superimposed portion is presented in a waveform as a total value of the first and second peak waveforms. The estimation part 102 estimates the entire waveform of a first peak, based, for example, on a waveform of a first portion of the first peak, to estimate a residual portion of the first peak waveform in the superimposed portion. The correction part 103 subtracts the residual portion of the first peak waveform from the actual waveform data. Note that the correction part 103 may subtract the entire waveform of the first peak from the actual waveform data. In this case, the actual waveform data is corrected so as to present waveform data of a second peak alone as illustrated in FIG. 2C.

Concrete example embodiments are described below in further detail with reference to drawings. Note that the same constituent components are denoted by the same reference signs, and descriptions thereof are omitted, in the example embodiments. Connecting lines between the blocks in each diagram include both bidirectional and unidirectional connecting lines. Each one-direction arrow is to schematically indicate a main flow of a signal (data) and is not intended to exclude bidirectional properties. In addition, an input port and an output port exist respectively at an input end and an output end of each connecting line although explicit illustrations thereof are omitted in circuit diagrams, block diagrams, inner configuration diagrams, connection diagrams, and the like illustrated in the disclosure of the present application. The same applies to an input/output interface.

### First Example Embodiment

A first example embodiment is described in more detail by using drawings.

In the first example embodiment, an electrophoresis apparatus that migrates fluorescence-labeled DNA chains is described.

In the disclosure of the present application, DNA chains to be subjected to electrophoresis are referred to as follows. The order in which DNA groups arrive, after electrophoresis is started, at a detection window is expressed using ordinal numbers. For example, assume that there exist two DNA groups provided with the same fluorescence label and having different sequence lengths (molecular weights). In this case, the DNA group arriving first at the detection window is referred to as a first DNA group, and the DNA group arriving later is referred to as a second DNA group.

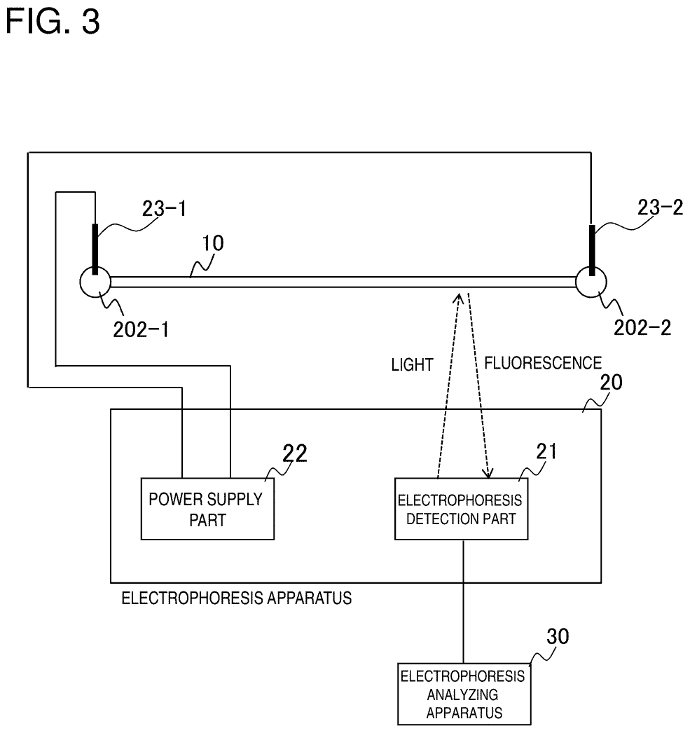

FIG. 3 is a diagram illustrating an example of a schematic configuration of an electrophoresis system according to the first example embodiment. In the first example embodiment, electrophoresis is performed using a capillary 10 illustrated in FIG. 3. Respective ends of the capillary 10 are connected to an electrode tank 202-1 and an electrode tank 202-2.

A sample including fluorescence-labeled DNA chains is injected into the capillary 10. Electrodes 23-1 and 23-2 are inserted into the electrode tanks 202-1 and 202-2, respectively.

The electrophoresis system also includes an electrophoresis apparatus 20 and an electrophoresis analyzing apparatus 30.

The electrophoresis apparatus 20 is an apparatus that performs electrophoresis by using the capillary 10. The electrophoresis apparatus 20 is formed by including an electrophoresis detection part 21 and a power supply part 22.

The electrophoresis detection unit 21 is a mechanism for detecting a fluorescence label. The electrophoresis detection part 21 includes, as a fluorescence label detection mechanism, an excitation device, such as an argon-ion laser, and a detection device, such as a filter or a camera.

The power supply part 22 is a means that applies an electrophoresis voltage to the capillary 10. More specifically, the power supply part 22 is connected to the electrodes 23-1 and 23-2 inserted into the respective electrode tanks 202-1 and 202-2. The power supply part 22 applies a direct voltage to the electrodes. Note that, upon starting of electrophoresis, the electrophoresis apparatus 20 notifies the electrophoresis analyzing apparatus 30 that electrophoresis is started.

When the direct voltage is applied to the electrodes 23 via the power supply part 22 and capillary electrophoresis is started, fluorescence-labeled DNA chains move from the electrode tank 202-1 in the direction toward the electrode tank 202-2. Upon starting of the electrophoresis, the electrophoresis detection part 21 monitors the capillary via the detection window to create actual waveform data indicating chronological changes in fluorescence brightness. The electrophoresis detection part 21 then outputs the created actual waveform data to the electrophoresis analyzing apparatus 30.

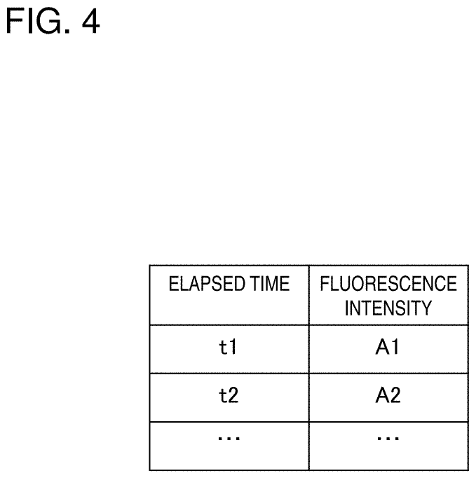

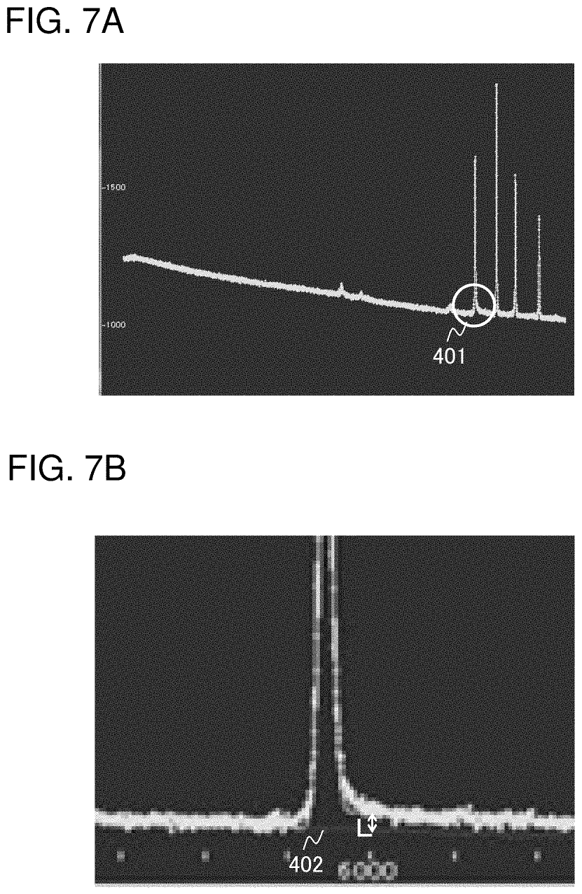

Specifically, the electrophoresis detection part 21 emits laser beams toward the capillary 10 via the detection window, and a fluorescent light at the detection window is received by an image sensor or the like. As illustrated in FIG. 4, the electrophoresis detection part 21 stores, in a storage medium (not illustrated), the brightness of a received fluorescent light in association with each time elapsed since the starting of the electrophoresis, and manages the association as a detection result. Note that the detection result is also expressed in the form of actual waveform data (refer to FIGS. 7A and 7B, for example) with the horizontal axis indicating to elapsed time and the vertical axis indicating fluorescence brightness. In the disclosure of the present application, a detection result in a digital form as illustrated in FIG. 4 is also referred to as actual waveform data.

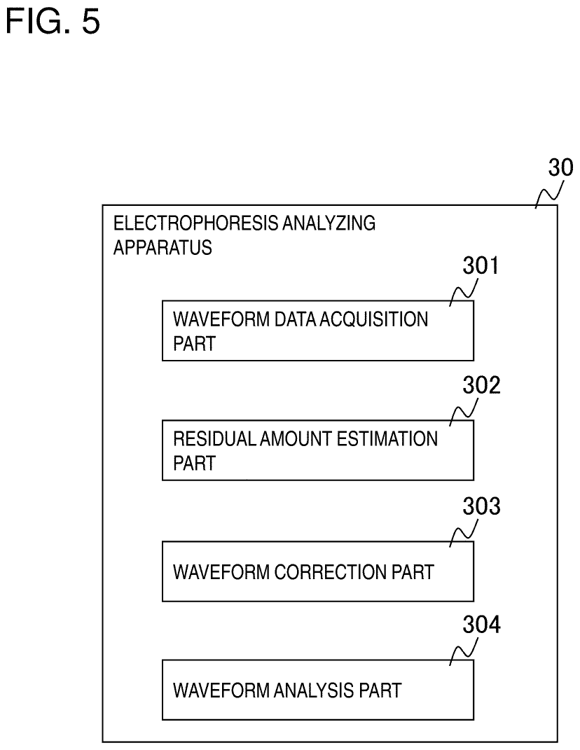

The electrophoresis detection part 30 analyzes the actual waveform data. FIG. 5 is a diagram illustrating an example of a configuration of the electrophoresis analyzing apparatus 30. As illustrated in FIG. 5, the electrophoresis analyzing apparatus 30 is configured by including a waveform data acquisition part 301, a residual amount estimation part 302, a waveform correction part 303, and a waveform analysis part 304.

The waveform data acquisition part 301 is a means that acquires actual waveform data from the electrophoresis apparatus 20. Specifically, the waveform data acquisition part 301 analyzes the actual waveform data acquired from the electrophoresis apparatus 20 to detect a peak waveform(s).

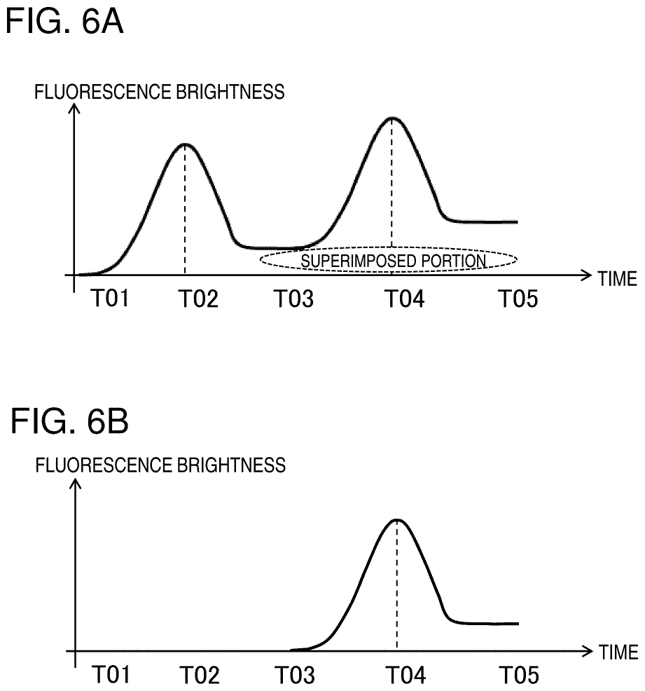

Conceptually, the waveform data acquisition part 301 acquires an actual waveform pattern as one illustrated in FIG. 6A. The actual waveform pattern illustrated in FIG. 6A illustrates a process in which DNA chains forming the first and second DNA groups move by migration. The first DNA group is expressed as a first peak waveform (waveform including the first peak) having time T02 as a center, and the second DNA group is expressed as a second peak waveform (waveform including the second peak) having time T04 as a center. The actual waveform pattern illustrated in FIG. 6A includes a superimposed portion of the first DNA group and the second DNA group from time T03 to time T05.

A reason why the above superimposed portion occurs is described below.

FIG. 7A is a diagram illustrating an example of a signal waveform (measured waveform) acquired through electrophoresis. FIG. 7B is an enlarged view of a region 401 in FIG. 7A.

With reference to FIG. 7B, it is confirmed that the waveform indicating changes in fluorescence brightness (referred to as “fluorescence waveform” below) is lifted from a baseline 402 after a peak time point. In other words, in FIG. 7B, an offset with a length L from the baseline 402 occurs after the peak time point.

Here, the fluorescence waveform is ideally assumed to have a Gaussian distribution shape. Specifically, in the example in FIG. 7B, the fluorescence waveform is assumed to converge on the baseline 402 after the peak time point. However, as described above, the actual fluorescence brightness has the offset with respect to the baseline 402 (deviation from the baseline 402 as a reference).

In view of this, a reason of the occurrence of the offset described above is considered.

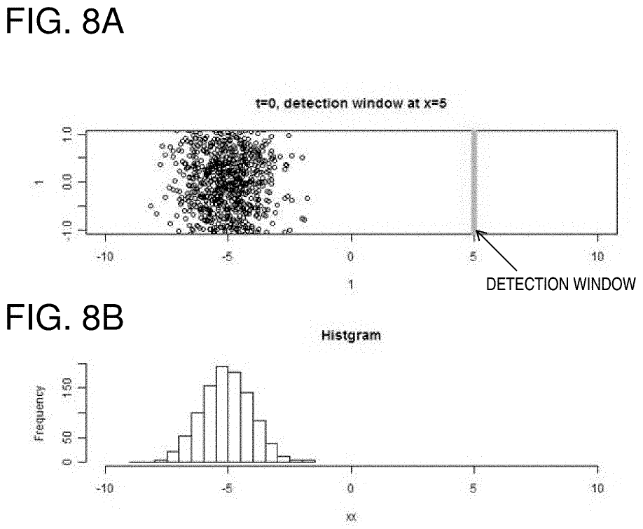

Assume that electrophoresis is performed using a flow path (capillary) as one illustrated in FIG. 8A. FIG. 8A illustrates a distribution of DNA chains immediately after the DNA chains are injected into the capillary. The position at which the DNA chains are injected is assumed to be X=−5, and, upon application of a direct voltage to the ends of the flow path, DNAs move from left to right. The measurement of fluorescent brightness is performed at the position of X=5. In FIG. 8A, a gap (detection window) for fluorescence detection is provided at the position of X=5. The distribution of the DNA chains immediately after the DNA chains are injected into the capillary is as illustrated in FIG. 8B. With reference to FIG. 8B, it is understood that the DNA chains are distributed with X=−5 as a center.

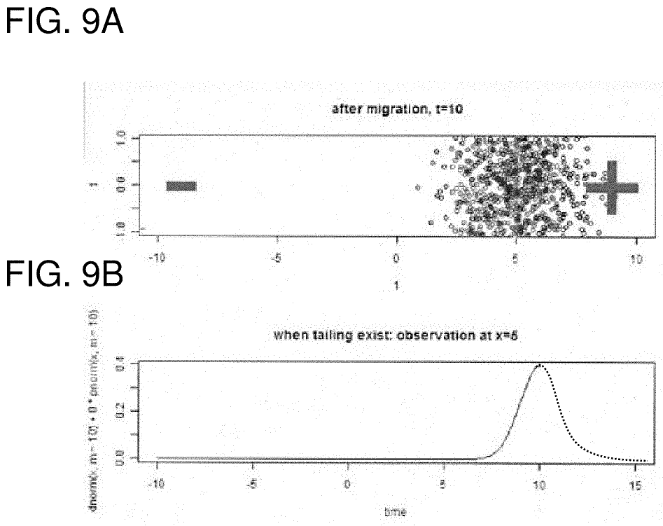

FIG. 9A illustrates a DNA distribution in a state where 10 seconds have elapsed since the application of the direct voltage to the ends of the flow path (a negative voltage to the left end, and a positive voltage to the right end). FIG. 9B illustrates a fluorescence waveform from the application of a direct voltage to the ends of the flow path to the elapse of 10 seconds. With reference to FIGS. 9A and 9B, the center of the fluorescence-labeled DNA group passes through the detection window, and the fluorescent brightness reaches the maximum (forms a peak), at time T=10. If all the injected DNAs thereafter pass through the detection window successfully, a fluorescence waveform as one illustrated with a dotted line in FIG. 9B is assumed to be acquired. Specifically, when the injected fluorescence-labeled DNA chains have similar moving speeds (substantially the same moving speed), a fluorescence waveform having a peak in a Gaussian distribution shape is assumed to be acquired.

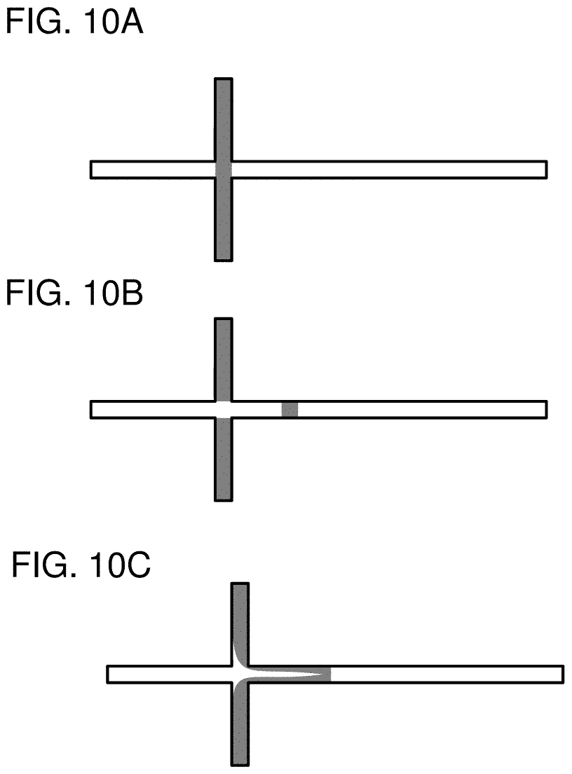

However, while DNA having the same sequence length are migrated at the same speed in theory, DNA are not migrated uniformly due to a diffusion phenomenon, such as Brownian motion, even having the same sequence length. In addition, as illustrated in FIG. 10A, for example, when a sample is injected into the capillary in a cross-injection method, electrophoresis is performed in a state where there still remain sample DNAs in the injection flow path. Here, ideally, only the sample DNAs at the position where the injection flow path and a capillary flow path cross is migrated as illustrated in FIG. 10B. However, in actuality, the sample DNAs remaining in the injection flow path are also drawn into the capillary flow path and move later, as illustrated in FIG. 10C. Note that, also in capillary electrophoresis, a phenomenon in which DNAs move later may occur due to polymer, buffer, or capillary contamination or the like.

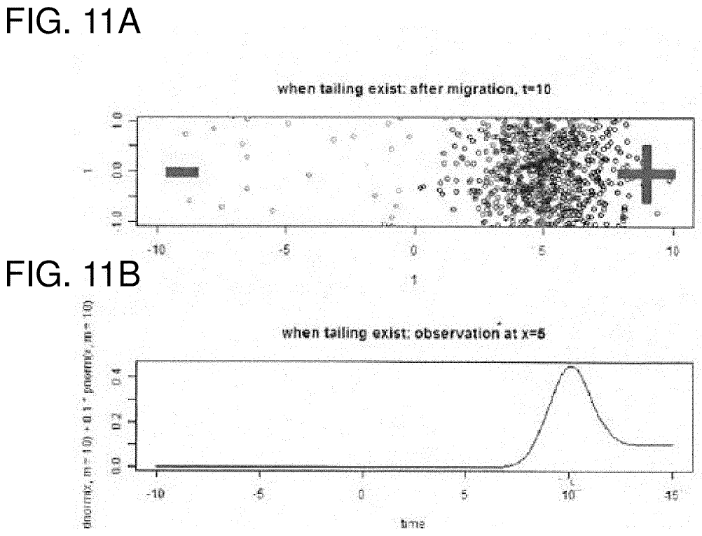

FIG. 11A illustrates a distribution of DNA chains in a state where 10 seconds have elapsed since application of a direct voltage to the ends of the flow path. FIG. 11B illustrates a fluorescence waveform from the application of the direct voltage to the ends of the flow path to the elapse of 15 seconds. With reference to FIG. 11A, although 10 seconds have already elapsed since the voltage application, fluorescence-labeled DNA chains still remain at X=−5 and X=0.

The residual DNAs result in arriving at the detection window (position of X=5) later than the other DNA chains. The DNA chains arriving later are also detected at the detection window, and consequently, a fluorescence waveform as one illustrated in FIG. 11B is obtained. In other words, the above-mentioned DNA chains arriving later are a cause of the offset having a length L illustrated in FIG. 7B.

Return the description to FIGS. 6A and 6B. Since part of the first DNA group forming the first peak waveform having time T02 as a center arrives at the detection window later than a greater part of the first DNA group, the fluorescence intensity in the latter portion of the first peak waveform does not reach zero. On the assumption that there constantly exists a certain quantity of such delayed DNA chains, the delayed DNA chains result in arriving at the detection window at the same time as the second DNA group forming the second peak waveform having time T04 as a center. In other words, the actual waveform data has a fluorescence waveform in which the second peak waveform and the residual portion of the first peak waveform (i.e., the delayed DNA chains) are superimposed on each other. Schematically, the delayed DNA chains cause the superimposed portion at time T03 to time T05 in FIG. 6A.

Return the description to FIG. 5. The residual amount estimation part 302 is a means that estimates, based on an already-appeared peak waveform, a residual portion of the already-appeared peak waveform in the superimposed portion, the already-appeared peak waveform having appeared before an analysis-target peak waveform to be subjected to waveform analysis. Here, the already-appeared peak waveform corresponds to the first peak waveform having time T02 as a center in FIGS. 6A and 6B, and the analysis-target peak waveform corresponds to the second peak waveform having time T04 as a center.

The residual amount estimation part 302 estimates the quantity of the delayed DNA chains in the first DNA group forming the first peak waveform, as a residual portion of the first peak waveform. The residual portion of the first peak waveform corresponds to the length L of the offset from the baseline 402 illustrated in FIGS. 7A and 7B.

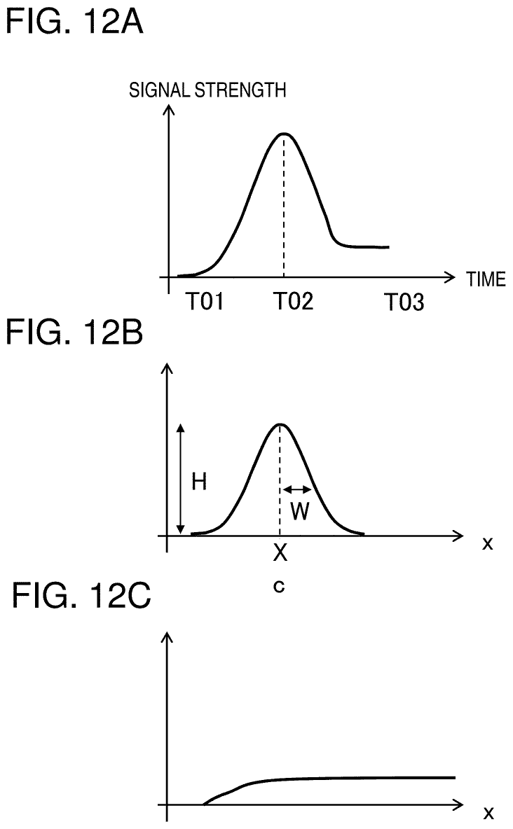

In a conceptual description, the residual amount estimation part 302 pays attention to the waveform at time T01 to time T03 in FIG. 6A, for the estimation of the residual portion. FIG. 12A is a graph illustrating part of the first peak waveform in FIG. 6A, the part corresponding to time T01 to time T03. The first peak waveform illustrated in FIG. 12A can be separated into a Gaussian waveform illustrated in FIG. 12B and a saturation waveform illustrated in FIG. 12C.

The Gaussian waveform illustrated in FIG. 12B is a fluorescence waveform derived from DNA chains assumed to have similar moving speeds. The Gaussian waveform illustrated in FIG. 12B can be modeled by Equation (1) below.

$$f 1 ( x ) = H * exp ( - ln ( 2 ) * {( \frac{x - Xc}{W} )}^{2} ) \qquad ( 1 )$$

In Equation (1), Xc denotes a center position of the Gaussian distribution, W denotes half-width at half-maximum (HWHM) of the Gaussian distribution, and H denotes the height of the Gaussian distribution (refer to FIG. 12B).

The saturation waveform illustrated in FIG. 12C is a fluorescence waveform derived from the residual portion of the first peak waveform (i.e., the delayed DNA chains).

On the assumption that variation in moving speed follows the Gaussian distribution, the saturation waveform is a similar figure to an “integral of the Gaussian function”. Note that, however, since not all of the DNAs (first DNA group) injected into the capillary 10 are delayed DNA chains, the integral of the Gaussian function is multiplied by a predetermined coefficient to approximate the waveform of signal strength brought about by delayed DNA chains (refer to FIG. 12C).

The waveform illustrated in FIG. 12C can be modeled by Equation (2) below.

$$f 2 ( x ) = \frac{α}{2} * ( 1 + erf ( sqrt ( ln ( 2 ) ) * ( \frac{x - Xc}{W} ) ) ) \qquad ( 2 )$$

Note that α denotes the predetermined coefficient by which the above-mentioned “integral of the Gaussian function” is multiplied. Moreover, erf denotes an error function, and sqrt is a function for obtaining a square root.

In this way, the first peak waveform illustrated in FIG. 12A is separated into the Gaussian waveform illustrated in FIG. 12B and the saturation waveform illustrated in FIG. 12C. In other words, the first peak waveform illustrated in FIG. 12A can be modeled by Equation (3) below.

f(x)=f1(x)+f2(x) (3)

According to Equation (3), it is understood that the waveform illustrated in FIG. 12A can be identified by four parameters (Xc, W, H, and α).

The residual amount estimation part 302 estimates the residual portion of the first peak waveform, based on the above viewpoints. Specifically, the residual amount estimation part 302 detects a peak waveform from the actual waveform data acquired by the waveform data acquisition part 301. In the example in FIG. 6A, the residual amount estimation part 302 detects a peak waveform having time T02 as a center.

The residual amount estimation part 302 then acquires data (fluorescence brightness) in a predetermined range having the detected peak as a center. For example, in the example in FIG. 6A, the residual amount estimation part 302 acquires fluorescence brightness values from time T01 to time T03 with time T02 as a center.

The residual amount estimation part 302 then identifies the four parameters (Xc, W, H, and a) defining the fluorescence waveform in the predetermined range, based on the data in the predetermined range having the detected peak as a center. Specifically, the residual amount estimation part 302 compares the detected peak waveform and a waveform obtained according to Equation (3) modeling the detected peak waveform, to calculate four parameters constituting Equation (3). For example, the residual amount estimation part 302 determines four parameters so that the difference between waveforms obtained by changing the four parameters and the corresponding actual waveform (waveform from time T01 to time T03 in FIG. 6A) would be minimum.

Upon determination of the four parameters, Equation (3) is determined. Moreover, Equation (2) is determined by using the four parameters. Equation (2) indicates the fluorescence brightness of the residual portion of the first peak waveform as illustrated in FIG. 12C.

In this way, the residual amount estimation part 302 models, by using Equation (3), waveform data as that illustrated from time T01 to time T03 in FIG. 6A. As a result of the modeling, four parameters characterizing each of Equations (1) and (2) are calculated. This can consequently derive Equation (2). Note that it is not possible to derive Equation (1) and Equation (2) individually at the time of modeling waveform data as that illustrated in FIG. 6A. This is because, as can be understood by referring to Equations (1) and (2), parameters characterizing the waveforms illustrated in FIG. 12B and FIG. 12C are in common.

The waveform correction part 303 is a means that subtracts a residual portion from actual waveform data to correct an analysis-target peak waveform to obtain a true analysis-target waveform. Specifically, the waveform correction part 303 subtracts the fluorescence brightness obtained based on the residual portion of the first peak waveform from the fluorescence intensity of the actual waveform data.

For example, in the example in FIG. 6A, the waveform correction part 303 subtracts the fluorescence brightness of the residual portion calculated according to Equation (2), from the fluorescence brightness from time T03 to time T05. The second peak waveform obtained as a result of this correction is a peak waveform from which the fluorescence brightness due to the residual portion of the first peak waveform is excluded, i.e., a true second peak waveform. For example, in the example in FIG. 6A, excluding the residual portion of the first peak waveform (i.e., the superimposed portion) results in the true second peak waveform illustrated in FIG. 6B.

The waveform analysis part 304 is a means that analyzes a true analysis-target waveform. For example, the waveform analysis part 304 calculates the area of a peak region included in a true analysis-target waveform to estimate a DNA amount. For example, with reference to FIG. 6B, it is considered that the waveform from time T03 to time T05 is a true analysis-target waveform obtained as a result of the correction by the waveform correction part 303. In view of this, the waveform analysis part 304 calculates the area of a region formed between the fluorescence brightness in the period from time T03 to time T05 and the elapsed time in the horizontal axis, to determine the area as the DNA amount of the second DNA group forming the second peak waveform.

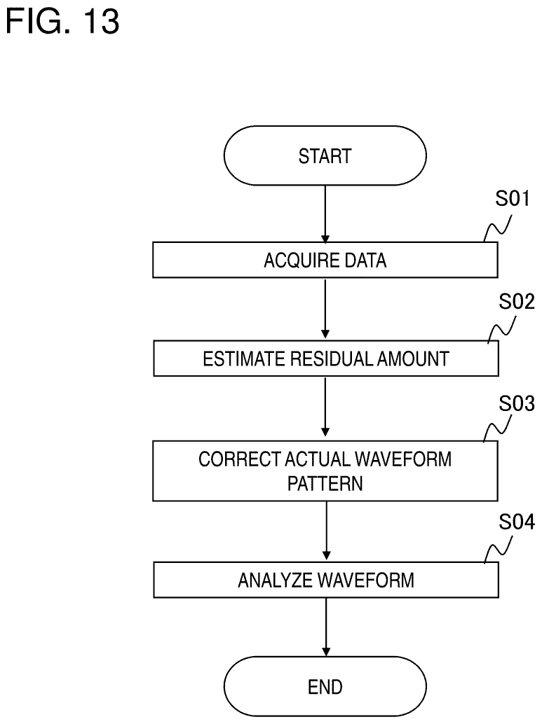

The summary of the operations of the electrophoresis analyzing apparatus 30 is as illustrated in FIG. 13.

In Step S01, the waveform data acquisition part 301 acquires a signal through electrophoresis.

In Step S02, the residual amount estimation part 302 estimates the residual amount of the first DNA group.

In Step S03, the waveform correction part 303 corrects an actual waveform pattern by using the estimated residual amount. Through the correction of the actual waveform pattern, a true analysis-target waveform is obtained.

In Step S04, the waveform analysis part 304 performs an analysis of the actual waveform pattern resulting from the correction.

A hardware configuration of the electrophoresis analyzing apparatus 30 according to the first example embodiment is described.

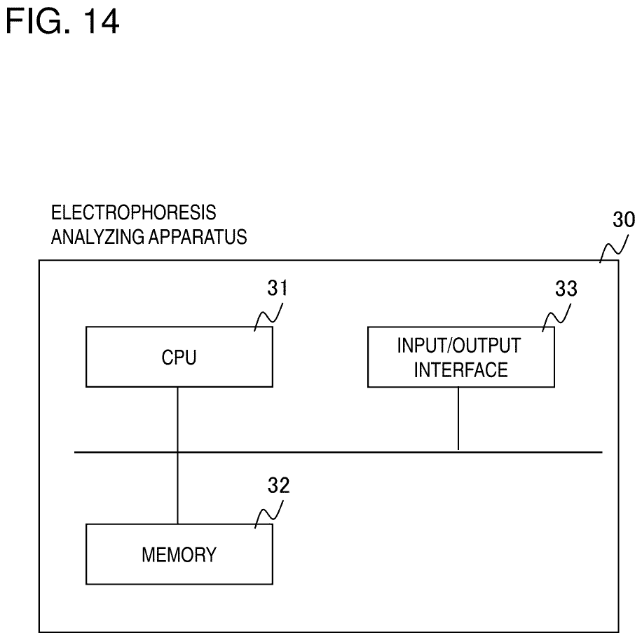

FIG. 14 is a block diagram illustrating an example of a hardware configuration of the electrophoresis analyzing apparatus 30 according to the first example embodiment. The electrophoresis analyzing apparatus 30 can be configured by a so-called computer (information processing apparatus) and includes a configuration illustrated in FIG. 14 as an example. For example, the electrophoresis analyzing apparatus 30 includes a central processing unit (CPU) 31, a memory 32, an input/output interface 33, and the like connected to each other through an internal bus.

Note that, however, the configuration illustrated in FIG. 14 is not intended to place any limitation on the hardware configuration of the electrophoresis analyzing apparatus 30. The electrophoresis analyzing apparatus 30 may include unillustrated hardware or may include a communication means as necessary, such as a network interface card (NIC). In addition, the number of CPUs and the like included in the electrophoresis analyzing apparatus 30 is not intended to be limited to the example in FIG. 14, and a plurality of CPUs may be included in the electrophoresis analyzing apparatus 30, for example.

The memory 32 is a random access memory (RAM), a read only memory (ROM), or an auxiliary storage (such as a hard disk).

The input/output interface 33 is an interface with an unillustrated display apparatus and/or input apparatus. The display apparatus is a liquid crystal display or the like, for example. The input apparatus is, for example, an apparatus that receives a user operation, such as a keyboard or a mouse, or an apparatus that inputs information from an external storage, such as a universal serial bus (USB) memory. A user inputs necessary information to the electrophoresis analyzing apparatus 30 by using a keyboard, a mouse, or the like. The input/output interface 33 also includes an interface (e.g., a USB interface) for connecting to the electrophoresis apparatus 20.

Functions of the electrophoresis analyzing apparatus 30 are implemented by the above-described processing modules. The processing modules are implemented, for example, by the CPU 31 executing a program stored in the memory 32. The program may be updated by downloading via a network or by using a storage medium having a program stored therein. Alternatively, the processing modules may be implemented with a semiconductor chip. In other words, the functions performed by the processing modules may be implemented using a kind of hardware and/or software. Moreover, a computer in which the above-described computer program is installed in a storage part thereof may be caused to function as the electrophoresis analyzing apparatus 30. Furthermore, by causing a computer to run the above-described program, an electrophoresis analysis method (a residual amount estimation method, a waveform correction method, a waveform analysis method, and the like) can be performed by the computer.

As described above, the electrophoresis analyzing apparatus 30 according to the first example embodiment estimates a residual amount of the first DNA group through analysis of an actual waveform pattern. By subtracting the residual amount estimated from an analysis-target actual waveform pattern, a more accurate analysis-target pattern can be obtained. Since residues of the first DNA group forming a peak first are eliminated from the analysis target thus obtained, more accurate analysis is possible.

The system configurations and operations described in the above example embodiments are examples, and various modifications are possible to be made. For example, the electrophoresis apparatus 20 and the electrophoresis analyzing apparatus 30 illustrated in FIG. 3 may be integrally formed.

In the above-described example embodiments, the operations of the electrophoresis analyzing apparatus 30 are described by using the waveform obtained based on the first and second DNA groups (waveform as that illustrated in FIG. 6A) as an example. However, a waveform to be input to the electrophoresis analyzing apparatus 30 may be one having two or more peaks. For example, electrophoresis is performed on four kinds of DNA, and an actual waveform pattern having four peaks may be an analysis target. In this case, a measured waveform of a third DNA group includes residues of first and second DNA groups. Hence, residues of the first and second DNA groups are estimated, and the residual amounts of the two DNA groups are subtracted from the measured waveform of the third DNA group, to thereby obtain a true analysis-target waveform.

A part or the whole of the above-described example embodiments can be described as, but is not limited to, the following modes.

### [Mode 1]

An electrophoresis analyzing apparatus according to the above-described first aspect.

### [Mode 2]

The electrophoresis analyzing apparatus according to Mode 1, in which

the estimation part compares the already-appeared peak waveform and a waveform according to a predetermined equation for modeling the already-appeared peak waveform and calculates a parameter(s) constituting the predetermined equation to thereby estimate the residual portion of the already-appeared peak waveform.

### [Mode 3]

The electrophoresis analyzing apparatus according to Mode 2, in which

the predetermine equation for modeling the already-appeared peak waveform is

$$f ( x ) = H * exp ( - ln ( 2 ) * {( \frac{x - Xc}{W} )}^{2} ) + \frac{α}{2} * ( 1 + erf ( sqrt ( ln ( 2 ) ) * ( \frac{x - Xc}{W} ) ) )$$

where Xc denotes a center position of a Gaussian distribution, W denotes a half-width at half-maximum of the Gaussian distribution, H denotes a height of the Gaussian distribution, and α denotes a predetermined coefficient.

### [Mode 4]

The electrophoresis analyzing apparatus according to Mode 3, in which

the estimation part determines a value calculated according to a following expression to be an estimation value of the residual portion.

$$\frac{α}{2} * ( 1 + erf ( sqrt ( ln ( 2 ) ) * ( \frac{x - Xc}{W} ) ) )$$

### [Mode 5]

The electrophoresis analyzing apparatus according to any one of Modes 1 to 4, further including a waveform analysis part configured to calculate an area of a peak region included in the true analysis-target waveform.

### [Mode 6]

The electrophoresis analyzing apparatus according to any one of Modes 1 to 5, in which the actual waveform data is data obtained through DNA capillary electrophoresis.

### [Mode 7]

The electrophoresis analyzing apparatus according to Mode 6, in which

the actual waveform data is DNA capillary electrophoresis by sample injection using a cross-injection method.

### [Mode 8]

An electrophoresis analysis method according to the above-described second aspect.

### [Mode 9]

A program according to the above-described third aspect.

Note that Mode 8 and Mode 9, as Mode 1, can be developed as in Modes 2 to 7.

Note that the disclosures in the above-mentioned cited patent literatures and the like are incorporated herein by reference. Making a change and adjustment of the example embodiments and examples is allowed within the framework of the entire disclosure (including the scope of the claims) of the present invention, and also based on a basic technical concept of the present invention. Further, various combinations or selections of various disclosed elements (including each element of each claim, each element of each example embodiment and each example, each element of each drawing, and the like) are allowed within the framework of the entire disclosure of the present invention. Specifically, as a matter of course, the present invention encompasses various modifications and amendments that may be achieved by a person skilled in the art based on the entire disclosure including the scope of the claims and the technical concept. Regarding a numerical range described herein, in particular, it should be interpreted that any numerical value or any smaller range included within the range is specifically described even without particular description.

### REFERENCE SIGNS LIST

10 Capillary
20 Electrophoresis apparatus
21 Electrophoresis detection part
22 Power supply part
23, 23-1, 23-2 Electrode
30, 100 Electrophoresis analyzing apparatus
31 Central processing unit (CPU)
32 Memory
33 Input/output interface
101 Acquisition part
102 Estimation part
103 Correction part
202-1, 202-2 Electrode tank
301 Waveform data acquisition part
302 Residual amount estimation part
303 Waveform correction part
304 Waveform analysis part
401 Region
402 Baseline

3. The electrophoresis analyzing apparatus according to claim 2, wherein
the predetermine equation for modeling the already-appeared peak waveform is
$$f ( x ) = H * exp ( - ln ( 2 ) * {( \frac{x - Xc}{W} )}^{2} ) + \frac{α}{2} * ( 1 + erf ( sqrt ( ln ( 2 ) ) * ( \frac{x - Xc}{W} ) ) )$$
where Xc denotes a center position of a Gaussian distribution, W denotes a half-width at half-maximum of the Gaussian distribution, H denotes a height of the Gaussian distribution, and α denotes a predetermined coefficient.

4. The electrophoresis analyzing apparatus according to claim 3, wherein
the estimation part determines a value calculated according to a following expression to be an estimation value of the residual portion.
$$\frac{α}{2} * ( 1 + erf ( sqrt ( ln ( 2 ) ) * ( \frac{x - Xc}{W} ) ) )$$

5. The electrophoresis analyzing apparatus according to claim 1, further comprising:
a waveform analysis part configured to calculate an area of a peak region included in the true analysis-target waveform.

6. The electrophoresis analyzing apparatus according to claim 1, wherein
the actual waveform data is data obtained through DNA capillary electrophoresis.

7. The electrophoresis analyzing apparatus according to claim 6, wherein
the actual waveform data is DNA capillary electrophoresis by sample injection using a cross-injection method.

8. An electrophoresis analysis method, comprising:
acquiring actual waveform data of electrophoresis, the actual waveform data including at least two peak waveforms partially including a superimposed portion;
estimating, from an already-appeared peak waveform, a residual portion of the already-appeared peak waveform in the superimposed portion, the already-appeared peak waveform having appeared, in the actual waveform data, before an analysis-target peak waveform to be subjected to waveform analysis; and
subtracting the residual portion from the superimposed portion to correct the analysis-target peak waveform to obtain a true analysis-target waveform.

9. A non-transitory computer-readable storage medium storing a program, the program causing a computer to execute:
acquiring actual waveform data of electrophoresis, the actual waveform data including at least two peak waveforms and partially including a superimposed portion;
estimating, from an already-appeared peak waveform, a residual portion of the already-appeared peak waveform in the superimposed portion, the already-appeared peak waveform having appeared, in the actual waveform data, before an analysis-target peak waveform to be subjected to waveform analysis; and
subtracting the residual portion from the superimposed portion and correcting the analysis-target peak waveform to obtain a true analysis-target waveform.

10. The electrophoresis analysis method according to claim 8, comprising:
comparing the already-appeared peak waveform and a waveform according to a predetermined equation for modeling the already-appeared peak waveform; and
calculating a parameter(s) constituting the predetermined equation to thereby estimate the residual portion of the already-appeared peak waveform.

11. The electrophoresis analysis method according to claim 10, wherein
the predetermine equation for modeling the already-appeared peak waveform is
$$f ( x ) = H * exp ( - ln ( 2 ) * {( \frac{x - Xc}{W} )}^{2} ) + \frac{α}{2} * ( 1 + erf ( sqrt ( ln ( 2 ) ) * ( \frac{x - Xc}{W} ) ) )$$
where Xc denotes a center position of a Gaussian distribution, W denotes a half-width at half-maximum of the Gaussian distribution, H denotes a height of the Gaussian distribution, and α denotes a predetermined coefficient.

12. The electrophoresis analysis method according to claim 11, wherein
the estimation part determines a value calculated according to a following expression to be an estimation value of the residual portion.
$$\frac{α}{2} * ( 1 + erf ( sqrt ( ln ( 2 ) ) * ( \frac{x - Xc}{W} ) ) )$$

13. The electrophoresis analysis method according to claim 8, further comprising:
calculating an area of a peak region included in the true analysis-target waveform.

14. The electrophoresis analysis method according to claim 8, wherein
the actual waveform data is data obtained through DNA capillary electrophoresis.

15. The electrophoresis analysis method according to claim 14, wherein
the actual waveform data is DNA capillary electrophoresis by sample injection using a cross-injection method.

16. The non-transitory computer-readable storage medium storing the program according to claim 9, the program causing a computer to execute:
comparing the already-appeared peak waveform and a waveform according to a predetermined equation for modeling the already-appeared peak waveform; and
calculating a parameter(s) constituting the predetermined equation to thereby estimate the residual portion of the already-appeared peak waveform.

17. The non-transitory computer-readable storage medium storing the program according to claim 16, wherein
the predetermine equation for modeling the already-appeared peak waveform is
$$f ( x ) = H * exp ( - ln ( 2 ) * {( \frac{x - Xc}{W} )}^{2} ) + \frac{α}{2} * ( 1 + erf ( sqrt ( ln ( 2 ) ) * ( \frac{x - Xc}{W} ) ) )$$
where Xc denotes a center position of a Gaussian distribution, W denotes a half-width at half-maximum of the Gaussian distribution, H denotes a height of the Gaussian distribution, and α denotes a predetermined coefficient.

18. The non-transitory computer-readable storage medium storing the program according to claim 17, wherein
the estimation part determines a value calculated according to a following expression to be an estimation value of the residual portion.
$$\frac{α}{2} * ( 1 + erf ( sqrt ( ln ( 2 ) ) * ( \frac{x - Xc}{W} ) ) )$$

19. The non-transitory computer-readable storage medium storing the program according to claim 9, the program causing a computer to execute:
calculating an area of a peak region included in the true analysis-target waveform.

20. The non-transitory computer-readable storage medium storing the program according to claim 9, wherein
the actual waveform data is data obtained through DNA capillary electrophoresis.
